SEQUENCE LISTING (17 sequences)

SEQ ID NO: 1 - protein, 13 aa, Artificial Sequence
  Synthetic L-CDR1 of anti-4-1BB antibody
SGSSSNIGNNYVT

SEQ ID NO: 2 - protein, 7 aa, Artificial Sequence
  Synthetic L-CDR2 of anti-4-1BB antibody
ADSHRPS

SEQ ID NO: 3 - protein, 11 aa, Artificial Sequence
  Synthetic L-CDR3 of anti-4-1BB antibody
ATWDYSLSGYV

SEQ ID NO: 4 - protein, 110 aa, Artificial Sequence
  Synthetic light chain variable region of anti-4-1BB antibody
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 5 - protein, 5 aa, Artificial Sequence
  Synthetic H-CDR1 of anti-4-1BB antibody
SYDMS

SEQ ID NO: 6 - protein, 17 aa, Artificial Sequence
  Synthetic H-CDR2 of anti-4-1BB antibody
WISYSGGSIYYADSVKG

SEQ ID NO: 7 - protein, 12 aa, Artificial Sequence
  Synthetic H-CDR3 of anti-4-1BB antibody
DAQRNSMREFDY

SEQ ID NO: 8 - protein, 121 aa, Artificial Sequence
  Synthetic heavy chain variable region of anti-4-1BB antibody
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 9 - protein, 450 aa, Artificial Sequence
  Synthetic heavy chain of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 719 aa, Artificial Sequence
  Synthetic heavy chain component of anti-4-1BB/anti-HER2 bispecific antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 11 - protein, 214 aa, Artificial Sequence
  Synthetic light chain of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 18 aa, Artificial Sequence
  Synthetic peptide linker
GSGSGSGSGSGSGSGSGS

SEQ ID NO: 13 - protein, 20 aa, Artificial Sequence
  Synthetic peptide linker
GGGGSGGGGSGGGGSGGGGS

SEQ ID NO: 14 - protein, 448 aa, Artificial Sequence
  Synthetic heavy chain  of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 121 aa, Artificial Sequence
  Synthetic heavy chain variable region of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSS

SEQ ID NO: 16 - protein, 216 aa, Artificial Sequence
  Synthetic light chain of anti-4-1BB antibody
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 17 - protein, 110 aa, Artificial Sequence
  Synthetic light chain variable region of anti-4-1BB antibody
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVL

(19) United States
(12) Patent Application Publication
(10) Pub. No.: US 2024/0084025 A1
(43) Pub. Date: Mar. 14, 2024

(54) METHODS FOR PURIFYING AN ANTI-4-1BB/ANTI-HER2 BISPECIFIC ANTIBODY

(71) Applicant: YUHAN CORPORATION, Seoul (KR)

(72) Inventors: Ju-Young PARK, Seoul (KR); Sung-Hyun CHOI, Suwon-si, Gyeonggi-do (KR); Yun-Hee CHOI, Yongin-si, Gyeonggi-do (KR); Won-Tae KIM, Suwon-si, Gyeonggi-do (KR)

(73) Assignee: YUHAN CORPORATION, Seoul (KR)

(21) Appl. No.: 18/273,888

(22) PCT Filed: Jan. 21, 2022
(86) PCT No.: PCT/KR2022/001083

§ 371 Date: Jul. 24, 2023

(30) Foreign Application Priority Data
Jan. 25, 2021 (KR) 10-2021-0010077

(51) Int. Cl. C07K 16/28; C07K 1/22; C07K 1/34; C07K 16/32

(52) U.S. Cl. CPC C07K 16/2878; C07K 1/22; C07K 1/34; C07K 16/32; C07K 2317/31; C07K 2317/56

(57) ABSTRACT

Provided is a method for purifying an anti-4-1BB/anti-HER2 bispecific antibody, the method of which includes carrying out affinity chromatography with a sodium acetate buffer containing a certain inorganic salt as an elution buffer. The purification method of the presently claimed subject matter increases the elution of an antibody in the intact form, thereby being able to provide an anti-4-1BB/anti-HER2 bispecific antibody in the intact form in high purity and high yield.

### TECHNICAL FIELD

The present invention relates to a method for purifying an anti-4-1BB/anti-HER2 bispecific antibody. More specifically, the present invention relates to a method for purifying an anti-4-1BB/anti-HER2 bispecific antibody, the method of which comprises carrying out affinity chromatography with a sodium acetate buffer containing a certain inorganic salt as an elution buffer.

### BACKGROUND ART

4-1BB protein is a member of TNF-receptor superfamily (TNFRSF) and is a co-stimulatory molecule which is expressed following the activation of immune cells, both innate and adaptive immune cells. 4-1BB plays important role in modulate the activity of various immune cells. 4-1BB agonists enhance proliferation and survival of immune cells, secretion of cytokines, and cytolytic activity CD8 T cells. Therefore, 4-1BB may be a promising target molecule in cancer immunology. Despite of their anti-tumor efficacy, anti-4-1BB antibody induced severe liver toxicity in clinical application.

HER2, which is a receptor tyrosine kinase (RTK) present on the surface of cells, induces proliferation and penetration of cancer cells, angiogenesis, etc.

Multispecific antibodies targeting two or more antigens are expected as a new drug having excellent therapeutic effects compared to a monoclonal antibody. A multispecific antibody capable of recognizing two different antigens wherein one is present on a cancer cell and the other is present on an immune cell can induce more potent cancer-specific immune responses. The present applicant has developed various antibodies that specifically bind to both 4-1BB and HER2, i.e., anti-4-1BB/anti-HER2 bispecific antibodies (International Patent Application No. PCT/KR2020/009871). The anti-4-1BB/anti-HER2 bispecific antibodies activate 4-1BB signaling and boost strong immune cells only in presence of HER2 expressing cells. Therefore, due to said specific HER2-mediated immune response, it is expected to have much less liver toxicity by using the bispecific antibodies compared to 4-1BB monoclonal antibody. The International Patent Application No. PCT/KR2020/009871 is incorporated herein by reference.

### DISCLOSURE

### Technical Problem

The present inventors recognized a problem that, when anti-4-1 BB/anti-HER2 bispecific antibody-producing cells were cultured and the anti-4-1BB/anti-HER2 bispecific antibodies were isolated therefrom according to the disclosures of International Patent Application No. PCT/KR2020/009871, the resulting antibodies in a significant amount are present in the form of aggregates. The present inventors carried out various studies to develop a purification method capable of increasing the purity of the anti-4-1BB/anti-HER2 bispecific antibody in the intact form. As the results thereof, it has been found that, when affinity chromatography is performed with a sodium acetate buffer containing a certain inorganic salt as an elution buffer, the anti-4-1BB/anti-HER2 bispecific antibody in the intact form can be obtained in high purity and high yield.

Therefore, it is an object of the present invention to provide a method for purifying an anti-4-1BB/anti-HER2 bispecific antibody, the method of which comprises carrying out affinity chromatography with a sodium acetate buffer containing a certain inorganic salt as an elution buffer.

Technical Solution

In accordance with an aspect of the present invention, there is provided a method for purifying an anti-4-1BB/anti-HER2 bispecific antibody, the method of which comprises (a) filtering a culture supernatant of an anti-4-1BB/anti-HER2 bispecific antibody-producing cell line to obtain a crude antibody-containing filtrate; (b) loading the filtrate onto a protein A affinity chromatography column; and (c) eluting the antibody from the column of step (b) with a sodium acetate buffer containing CaCl₂.

In the purification method of the present invention, the heavy chain of the anti-4-1BB/anti-HER2 bispecific antibody comprises (i) a heavy chain of anti-HER2 antibody consisting of the amino acids of SEQ ID NO: 9 and (ii) a scFv of anti-4-1BB antibody comprising a light chain variable region of anti-4-1BB antibody comprising the amino acids of SEQ ID NOs: 1, 2, and 3; and a heavy chain variable region of anti-4-1BB antibody comprising the amino acids of SEQ ID NOs: 5, 6, and 7, and the light chain of the anti-4-1BB/anti-HER2 bispecific antibody comprises a light chain of anti-HER2 antibody consisting of the amino acids of SEQ ID NO: 11.

In an embodiment, the light chain variable region of anti-4-1BB antibody may consist of the amino acids of SEQ ID NO: 4; and the heavy chain variable region of anti-4-1BB antibody may consist of the amino acids of SEQ ID NO: 8. In another embodiment, the scFv of anti-4-1BB antibody may further comprise a linker consisting of the amino acids of SEQ ID NO: 13. In still another embodiment, the heavy chain of the anti-4-1BB/anti-HER2 bispecific antibody may further comprise (iii) a linker consisting of the amino acids of SEQ ID NO: 12. In still another embodiment, the heavy chain of the anti-4-1BB/anti-HER2 bispecific antibody may consist of the amino acids of SEQ ID NO:

In the purification method of the present invention, CaCl₂ may be present in a concentration ranging from 30 to 500 mM, preferably from 75 to 125 mM, in the sodium acetate buffer. And, sodium acetate may be present in a concentration ranging from 20 to 100 mM in the sodium acetate buffer. In addition, the sodium acetate buffer may have a pH ranging from pH 3.5 to pH 4.0.

### Advantageous Effects

When affinity chromatography is performed with a sodium acetate buffer containing a certain inorganic salt, i.e., CaCl₂, as an elution buffer according to the present invention, the elution of an antibody in the intact form can be remarkably increased, thereby being able to provide an anti-4-1BB/anti-HER2 bispecific antibody in the intact form in high purity and high yield. Therefore, the purification method of the present invention can provide the anti-4-1BB/anti-HER2 bispecific antibody in high purity and high yield.

### BEST MODE

The present invention provides a method for purifying an anti-4-1BB/anti-HER2 bispecific antibody, the method of which comprises (a) filtering a culture supernatant of an anti-4-1BB/anti-HER2 bispecific antibody-producing cell line to obtain a crude antibody-containing filtrate; (b) loading the filtrate onto a protein A affinity chromatography column; and (c) eluting the antibody from the column of step (b) with a sodium acetate buffer containing CaCl₂.

In the purification method of the present invention, the anti-4-1 BB/anti-HER2 bispecific antibody consists of a heavy chain and a light chain, which may be prepared as disclosed in International Patent Application No. PCT/KR2020/009871. Specifically, the heavy chain of the anti-4-1BB/anti-HER2 bispecific antibody comprises (i) a heavy chain of anti-HER2 antibody consisting of the amino acids of SEQ ID NO: 9 and (ii) a scFv (single-chain Fv) of anti-4-1BB antibody comprising a light chain variable region of anti-4-1BB antibody comprising the amino acids of SEQ ID NOs: 1, 2, and 3; and a heavy chain variable region of anti-4-1BB antibody comprising the amino acids of SEQ ID NOs: 5, 6, and 7, and the light chain of the anti-4-1BB/anti-HER2 bispecific antibody comprises a light chain of anti-HER2 antibody consisting of the amino acids of SEQ ID NO: 11.

In an embodiment, the light chain variable region of anti-4-1BB antibody may consist of the amino acids of SEQ ID NO: 4 and the heavy chain variable region of anti-4-1BB antibody may consist of the amino acids of SEQ ID NO: 8. In another embodiment, the scFv of anti-4-1BB antibody may further comprise a linker consisting of the amino acids of SEQ ID NO: 13. For example, the scFv of anti-4-1BB antibody may be a polypeptide in which the amino acids of SEQ ID NO: 4, the amino acids of SEQ ID NO: 13, and the amino acids of SEQ ID NO: 8 are linked in sequence (i.e., N′->C′).

In an embodiment, the heavy chain of the anti-4-1BB/anti-HER2 bispecific antibody may further comprise (iii) a linker consisting of the amino acids of SEQ ID NO: 12. For example, the heavy chain of the anti-4-1BB/anti-HER2 bispecific antibody may be a polypeptide in which the amino acids of SEQ ID NO: 9, the amino acids of SEQ ID NO: 12, the amino acids of SEQ ID NO: 4, the amino acids of SEQ ID NO: 13, and the amino acids of SEQ ID NO: 8 are linked in sequence (i.e., N′->C′), i.e., a polypeptide consisting of the amino acids of SEQ ID NO: 10.

The purification method of the present invention includes filtering a culture supernatant of an anti-4-1BB/anti-HER2 bispecific antibody-producing cell line to obtain a filtrate containing a crude antibody [step (a)]. The anti-4-1BB/anti-HER2 bispecific antibody-producing cell line may be cells (e.g., CHO cells) transfected with the plasmid(s) into which polynucleotides encoding the heavy and light chains of the anti-4-1BB/anti-HER2 bispecific antibody respectively are inserted. Said transfected cells may be obtained as disclosed in International Patent Application No. PCT/KR2020/009871. The transfected cells may be cultured in a medium conventionally used in the field of biotechnology according to a conventional method. The culture supernatant may be obtained e.g., by centrifuging a culture of the anti-4-1BB/anti-HER2 bispecific antibody-producing cell line. The filtering may be carried out according to a conventional aseptic filtration, e.g., an aseptic filtration through a 0.2 μm membrane filter. The crude antibody-containing filtrate obtained as described above includes about 40 to 45% of antibody aggregates.

The purification method of the present invention includes loading the filtrate obtained in step (a) onto a protein A affinity chromatography column [step (b)]. The protein A affinity chromatography includes the use of various agarose-based resins. As the resin, there may be used known agarose-based resins, for example, affinity chromatography resins such as MabSelect SuRe™ LX (Cytiva), MabSelect™ PrismA (Cytiva), Praesto™ jetted A50 (Purolite Life Sciences), etc., preferably MabSelect™ PrismA (Cytiva). Loading the filtrate obtained in step (a) onto a protein A affinity chromatography column filled with e.g., MabSelect™ PrismA (Cytiva) resins may be carried out according to a conventional method. For example, the filtrate may be loaded at a rate of 20 to 40 mg per ml of resin, but is not limited thereto.

The purification method of the present invention includes eluting the antibody (i.e., an anti-4-1BB/anti-HER2 bispecific antibody) from the column of step (b) with a sodium acetate buffer containing CaCl₂ as an elution buffer [step (c)]. The CaCl₂ may be present preferably in a concentration ranging from 30 to 500 mM, more preferably in a concentration ranging from 50 to 500 mM, still more preferably in a concentration ranging from 75 to 125 mM, particularly preferably in a concentration of about 100 mM, in the sodium acetate buffer. The sodium acetate may be present preferably in a concentration ranging from 20 to 100 mM in the sodium acetate buffer. In an embodiment of the present invention, CaCl₂ may be present in a concentration ranging from 75 to 125 mM in the sodium acetate buffer and sodium acetate may be present in a concentration ranging from 20 to 100 mM in the sodium acetate buffer.

The sodium acetate buffer may have preferably a pH ranging from pH 3.5 to pH 4.0, more preferably a pH of about pH 3.7. In an embodiment of the present invention, CaCl₂ may be present in a concentration ranging from 75 to 125 mM in the sodium acetate buffer; sodium acetate may be present in a concentration ranging from 20 to 100 mM in the sodium acetate buffer; and the sodium acetate buffer may have a pH ranging from pH 3.5 to pH 4.0.

In an embodiment of the purification method according to the present invention, the sodium acetate buffer may be a 100 mM sodium acetate buffer containing about 100 mM CaCl₂) and having a pH of about pH 3.7.

In another embodiment of the purification method according to the present invention, the sodium acetate buffer may be a 20 mM sodium acetate buffer containing about 100 mM CaCl₂ and having a pH of about pH 3.7.

The anti-4-1BB/anti-HER2 bispecific antibody obtained according to the purification method of the present invention may be isolated in the form of an antibody-containing solution from the eluted solution of step (c) by carrying out virus inactivation and neutralization processes according to conventional methods. The virus inactivation may be carried out, e.g., by adjusting the pH to a pH of 3.4 to 3.6 and the neutralization may be carried out, e.g., by using a 1M tromethamine solution, but is not limited thereto. If necessary, after performing the process of step (c), there may be additionally carried out one or more chromatography selected from the group consisting of cation exchange chromatography, anion exchange chromatography, mixed-mode chromatography, and hydrophobic interaction chromatography.

The present invention will be described in further detail with reference to the following preparation examples and examples. These preparation examples and examples are for illustrative purposes only and are not intended to limit the scope of the present invention.

### Preparation Example: Preparation of Anti-4-1BB/Anti-HER2 Bispecific Antibody

In accordance with the methods disclosed in International Patent Application No. PCT/KR2020/009871, an anti-4-1BB/anti-HER2 bispecific antibody was prepared as follows.

(1) Anti-4-1BB scFv Antibody

Screening was carried out by phage library immunotube panning against 4-1BB. For panning of the phage library (obtained from KBio Health and CUREBIO) against the target molecule, four rounds of panning were carried out in total using human 4-1BB (NCBI Accession No. NP_001552.2) coated immunotubes.

Bacterial colonies from the 3 rounds of panning output were grown in SB-Carbenicillin (Biomatik cat #A2311-5g) in 96 deepwell plate until turbid, at which point 10¹¹ pfu of VCSM13 helper phage (K-Bio Health) was added to each well. After 1 hour infection at 37° C. with gentle shaking (80 rpm), 70 μg/mL of kanamycin was added and the cells were cultured overnight at 30° C. with shaking at 200 rpm.

Next day, the plates were centrifuged and the supernatants containing the phages were added to 4-1BB antigen-coated ELISA plates blocked with 3% (v/v) BSA (bovine serum albumin) in PBST (Phosphate Buffered Saline with Tween 20). After 1 hour incubation at room temperature, the plates were washed three times with PBST and anti-M13 antibody (Sino Biological cat #11973-MM05) was added. The plates were incubated for 1 hour, washed three times with PBST, and the binding activity was measured using tetramethylbenzidine (TMB).

The 4-1BB specific binders were amplified for plasmid DNA sequencing. The light chain- and heavy chain-variable region (VL and VH) sequences were analyzed to identify unique sequences and determine sequence diversity. The anti-4-1BB antibody indicated as BMUR (BMS's Urelumab, U.S. Pat. No. 7,288,638) is used for comparing agonistic activity. As the results thereof, the sequences of the full human monoclonal antibody against 4-1BB are as follows.

TABLE 1

| | Amino acid sequences (N′→C′) |
|---|---|
| Heavy chain | EVQLLESGGGLVQPGGSLRLSCAASGFTFSSYDMSWVRQAPGKGLE |
| | WVSWISYSGGSIYYADSVKGRFTISRDNSKNTLYLQMNSLRAEDTAV |
| | YYCARDAQRNSMREFDYWGQGTLVTVSSASTKGPSVFPLAPCSRST |
| | SESTAALGCLVKDYFPEPVTVSWNSGALTSGVHTFPAVLQSSGLYSL |
| | SSVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSN |
| | KGLPSSIEKTISKAKGQPREPQVYTLPPSQEEMTKNQVSLTCLVKGFY |
| | PSDIAVEWESNGQPENNYKTTPPVLDSDGSFFLYSRLTVDKSRWQE |
| | GNVFSCSVMHEALHNHYTQKSLSLSLGK (SEQ ID NO: 14) |
| Heavy chain | EVQLLESGGGLVQPGGSLRLSCAASGFTFSSYDMSWVRQAPGKGLE |
| variable region | WVSWISYSGGSIYYADSVKGRFTISRDNSKNTLYLQMNSLRAEDTAV |
| (VH) | YYCARDAQRNSMREFDYWGQGTLVTVSS (SEQ ID NO: 15) |
| H-CDR1 | SYDMS (SEQ ID NO: 5) |
| H-CDR2 | WISYSGGSIYYADSVKG (SEQ ID NO: 6) |
| H-CDR3 | DAQRNSMREFDY (SEQ ID NO: 7) |
| Light chain | QSVLTQPPSASGTPGQRVTISCSGSSSNIGNNYVTWYQQLPGTAPKL |
| | LIYADSHRPSGVPDRFSGSKSGTSASLAISGLRSEDEADYYCATWDY |
| | SLSGYVFGGGTKLTVLGQPKAAPSVTLFPPSSEELQANKATLVCLISD |
| | FYPGAVTVAWKADSSPVKAGVETTTPSKQSNNKYAASSYLSLTPEQ |
| | WKSHRSYSCQVTHEGSTVEKTVAPAECS (SEQ ID NO: 16) |
| Light chain | QSVLTQPPSASGTPGQRVTISCSGSSSNIGNNYVTWYQQLPGTAPKL |
| variable region | LIYADSHRPSGVPDRFSGSKSGTSASLAISGLRSEDEADYYCATWDY |
| (VL) | SLSGYVFGGGTKLTVL (SEQ ID NO: 17) |
| L-CDR1 | SGSSSNIGNNYVT (SEQ ID NO: 1) |
| L-CDR2 | ADSHRPS (SEQ ID NO: 2) |
| L-CDR3 | ATWDYSLSGYV (SEQ ID NO: 3) |

The anti-4-1BB scFv antibody with a structure of (N′)-VL-linker-VH-(C′) shown in Table 2 below was prepared using (GGGGS)₄ of SEQ ID NO: 13 as a linker, wherein the amino acid residue “G” at the position 103 of the light chain variable region was substituted with “C” (SEQ ID NO: 4) and the amino acid residue “G” at the position 44 of a heavy chain variable region was substituted with “C” (SEQ ID NO: 8), in order to stabilize the scFv by generating a disulfide bridge. Specifically, the polynucleotide in which the DNA encoding the light chain variable region of SEQ ID NO: 4, the DNA encoding the linker of SEQ ID NO: 13, and the DNA encoding the heavy chain variable region of SEQ ID NO: 8 are linked was inserted into pcDNA 3.4 (Invitrogen, A14697). The resulting vector was transfected into ExpiCHO cells (Gibco, A29127) at 37° C. using ExpiFectamine™ CHO Kit (Gibco, A29133), followed by culturing the cells for 8 days. The culture supernatant was filtered with a 0.2 μm filter to prepare an anti-4-1BB scFv antibody with a structure of (N′)-VL-linker-VH-(C′).

TABLE 2

| | Amino acid sequences |
|---|---|
| | (N′→C′) |
| Light | QSVLTQPPSASGTPGQRVTISC |
| chain | SGSSSNIGNNYVTWYQQLPGTA |
| variable | PKLLIYADSHRPSGVPDRFSGS |
| region | KSGTSASLAISGLRSEDEADYY |
| (VL) | CATWDYSLSGYVFGCGTKLTVL |
| | (SEQ ID NO: 4) |
| Linker | GGGGSGGGGSGGGGSGGGGS |
| | (SEQ ID NO: 13) |
| Heavy | EVQLLESGGGLVQPGGSLRLSC |
| chain | AASGFTFSSYDMSWVRQAPGKC |
| variable | LEWVSWISYSGGSIYYADSVKG |
| region | RFTISRDNSKNTLYLQMNSLRA |
| (VH) | EDTAVYYCARDAQRNSMREFDY |
| | WGQGTLVTVSS |
| | (SEQ ID NO: 8) |

(2) Anti-HER2 Antibody

As HER2 targeting moieties for an anti-4-1BB/anti-HER2 bispecific antibody, the heavy and light chains of trastuzumab (Genentech; hereinafter referred to as “HER2 (WT)”, DrugBank Accession No. DB00072; human IgG1 Kappa monoclonal antibody) were used. The amino acid sequences of the heavy and light chains of HER2 (WT) are shown in Table 3 below.

TABLE 3

| HER2 (WT) | Amino acid sequences (N′→C′) |
|---|---|
| Heavy chain | EVQLVESGGGLVQPGGSLRLSCAASGFNIK |
| | DTYIHWVRQAPGKGLEWVARIYPTNGYTRY |
| | ADSVKGRFTISADTSKNTAYLQMNSLRAED |
| | TAVYYCSRWGGDGFYAMDYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPELLGG |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSREE |
| | MTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGK |
| | (SEQ ID NO: 9) |
| Light chain | DIQMTQSPSSLSASVGDRVTITCRASQDVN |
| | TAVAWYQQKPGKAPKLLIYSASFLYSGVPS |
| | RFSGSRSGTDFTLTISSLQPEDFATYYCQQ |
| | HYTTPPTFGQGTKVEIKRTVAAPSVFIFPP |
| | SDEQLKSGTASVVCLLNNFYPREAKVQWKV |
| | DNALQSGNSQESVTEQDSKDSTYSLSSTLT |
| | LSKADYEKHKVYACEVTHQGLSSPVTKSFN |
| | RGEC (SEQ ID NO: 11) |

(3) Anti-4-1BB/Anti-HER2 Bispecific Antibody-Producing Cell Line

Using the anti-4-1BB scFv antibody and the heavy and light chains of HER2 (WT) obtained in (1) and (2) above, we prepared an antibody in the IgG-scFv fusion form (the scFv antibody fragment is fused to the c-terminus of IgG), i.e., an anti-4-1BB/anti-HER2 bispecific antibody having a heavy component consisting of SEQ ID NO: 10 and a light component consisting of SEQ ID NO: 11, as shown in Table 4 below.

Specifically, the DNA segment 2 encoding the GS linker of SEQ ID NO: 12 was fused to the terminus of the DNA segment 1 encoding the heavy chain of anti-HER2 antibody (SEQ ID NO: 9); and then the DNA segment 3 encoding the scFv of anti-4-1BB antibody was fused thereto to prepare a DNA segment encoding the heavy chain of anti-HER2/anti-4-1BB bispecific antibody (SEQ ID NO: 10), which was then inserted into a plasmid 1 (Invitrogen, A14697). In addition, a DNA segment 4 encoding the light chain of anti-HER2/anti-4-1BB bispecific antibody (SEQ ID NO: 11) was inserted into a plasmid 2 (Invitrogen, A14697).

The resulting plasmids 1 and 2 were transfected into ExpiCHO cells (Gibco, A29127) using the ExpiCHO™ Expression System (Gibco, A29133). Specifically, the obtained plasmid 1 (250 μg) and 2 (250 μg) were mixed with OptiPRO™ SFM (12309-050) with ExpiFectamine CHO reagent (A29129) (800 μL) (final volume: 20 mL), followed by being left for 3 minutes. For transfection, the resulting mixed solution and 6×10⁶ ExpiCHO cells were cultured in a ExpiCHO expression medium (A29100-01) in an 8% CO₂ humidified shaking incubator at 37° C. After 18 hours of incubation, 1.5 mL of ExpiFectamine™ CHO Transfection Enhancer 1 and ExpiFectamine™ CHO Feed were added thereto and then the incubation was continued at 37° C. for 4 days. After the temperature was adjusted to 32° C., ExpiFectamine™ CHO Feed was added thereto and then the incubation was continued for 8 days.

TABLE 4

| | | | Amino acid sequences (N′→C′) |
|---|---|---|---|
| Heavy | \textcircled{1} Heavy chain | | EVQLVESGGGLVQPGGSLRLSCAASGFNIKDTYIH |
| | of anti-HER2 | | WVRQAPGKGLEWVARIYPTNGYTRYADSVKGRF |
| | antibody | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNV |
| | | | NHKPSNTKVDKKVEPKSCDKTHTCPPCPAPELLG |
| | | | GPSVFLFPPKPKDTLMISRTPEVTCVVVDVSHEDP |
| | | | EVKFNWYVDGVEVHNAKTKPREEQYNSTYRVVS |
| | | | VLTVLHQDWLNGKEYKCKVSNKALPAPIEKTISKA |
| | | | KGQPREPQVYTLPPSREEMTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKS |
| | | | LSLSPGK (SEQ ID NO: 9) |
| component | \textcircled{2} Linker | | GSGSGSGSGSGSGSGSGS (SEQ ID NO: 12) |
| | \textcircled{3} scFv | VL | QSVLTQPPSASGTPGQRVTISCSGSSSNIGNNYVT |
| | of | | WYQQLPGTAPKLLIYADSHRPSGVPDRFSGSKSG |
| | anti- | | TSASLAISGLRSEDEADYYCATWDYSLSGYVFGC |
| | 4-1BB | | GTKLTVL (SEQ ID NO: 4) |
| | antibody | | * L-CDR1: SGSSSNIGNNYVT (SEQ ID NO: 1) |
| | | | * L-CDR2: ADSHRPS (SEQ ID NO: 2) |
| | | | * L-CDR3: ATWDYSLSGYV (SEQ ID NO: 3) |
| | | Linker | GGGGGGGGSGGGGSGGGGS (SEQ ID NO: 13) |
| | | VH | EVQLLESGGGLVQPGGSLRLSCAASGFTFSSYDM |

| | | |
|---|---|---|
| | | SWVRQAPGKCLEWVSWISYSGGSIYYADSVKGR |
| | | FTISRDNSKNTLYLQMNSLRAEDTAVYYCARDAQ |
| | | RNSMREFDYWGQGTLVTVSS (SEQ ID NO: 8) |
| | | * H-CDR1: SYDMS (SEQ ID NO: 5) |
| | | * H-CDR2: WISYSGGSIYYADSVKG (SEQ ID NO: 6) |
| | | * H-CDR3: DAQRNSMREFDY (SEQ ID NO: 7) |
| | Heavy | EVQLVESGGGLVQPGGSLRLSCAASGFNIKDTYI |
| | component | HWVRQAPGKGLEWVARIYPTNGYTRYADSVKGRF |
| | (\textcircled{1}+ \textcircled{2} + \textcircled{3}) | TISADTSKNTAYLQMNSLRAEDTAVYYCSRWGGD |
| | | GFYAMDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHE |
| | | DPEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFF |
| | | LYSKLTVDKSRWQQGNVFSCSVMHEALHNHYTQK |
| | | SLSLSPGKGSGSGSGSGSGSGSGSGSQSVLTQPP |
| | | SASGTPGQRVTISCSGSSSNIGNNYVTWYQQLPG |
| | | TAPKLLIYADSHRPSGVPDRFSGSKSGTSASLAI |
| | | SGLRSEDEADYYCATWDYSLSGYVFGCGTKLTVL |
| | | GGGGSGGGGSGGGGSGGGGSEVQLLESGGGLVQ |
| | | PGGSLRLSCAASGFTFSSYDMSWVRQAPGKCLE |
| | | WVSWISYSGGSIYYADSVKGRFTISRDNSKNTL |
| | | YLQMNSLRAEDTAVYYCARDAQRNSMREFDYWG |
| | | QGTLVTVSS (SEQ ID NO: 10) |
| Light | Light chain of | DIQMTQSPSSLSASVGDRVTITCRASQDVNTAVA |
| component | anti-HER2 | WYQQKPGKAPKLLIYSASFLYSGVPSRFSGSRSG |
| | antibody | TDFTLTISSLQPEDFATYYCQQHYTTPPTFGQGTK |
| | | VEIKRTVAAPSVFIFPPSDEQLKSGTASVVCLLNN |
| | | FYPREAKVQWKVDNALQSGNSQESVTEQDSKDSTY |
| | | SLSSTLTLSKADYEKHKVYACEVTHQGLSSPVTK |
| | | SFNRGEC (SEQ ID NO: 11) |

(4) Fed-Batch Culture for Antibody Expression

After culturing the anti-4-1BB/anti-HER2 bispecific antibody-producing cell line obtained in (3) in a glutamine-free Actipro medium (Hyclone, SH31037.02) for 3 days, fed-batch culture (working volume: 700 ml/2000 ml flask, 37° C., 5% CO₂, 130 rpm) was carried out for 14 days, while Cell Boost™ 7a (Hyclone, SH31026.01)/7b supplements (Hyclone, SH31027.01KR) were added every other day. The culture supernatant obtained by centrifuging the culture at about 3000 rpm was filtered with a 0.2 μm membrane filter to obtain a crude antibody-containing filtrate.

### Example 1: Purification of Anti-4-1BB/Anti-HER2 Bispecific Antibody by Protein a Affinity Chromatography

MabSelect PrismA affinity chromatography resin (Cytiva) was filled into a column having 1.6 cm of diameter and 20 cm of bed height to prepare a protein A affinity chromatography column, which was then equilibrated with 1× phosphate buffered saline (PBS) (pH 7.4). The crude antibody-containing filtrate obtained in Preparation Example (4) was loaded onto the protein A affinity chromatography column at a ratio of 30 mg per mL of resin. After washing the column with 1×PBS (pH 7.4), the antibody was eluted with the sodium acetate buffers containing an excipient (CaCl₂) or with the sodium acetate buffer having no excipient as an elution buffer. The concentrations of excipient and sodium acetate in the elution buffer and the pH of the elution buffer are shown in Table 5 below.

For the pools obtained using the respective elution buffers, the yields of the antibody (i.e., anti-4-1 BB/anti-HER2 bispecific antibody) were analyzed by measuring UV at 280 nm; and the purities of the antibody (i.e., anti-4-1BB/anti-HER2 bispecific antibody) were analyzed by measuring the contents (%) of high molecular weight aggregates using size exclusion chromatography (SEC-HPLC). The analysis using the SEC-HPLC was carried out using a TOSOH model TSK-GEL G3000SW_{XL} column.

After equilibrating the column by flowing 1× phosphate buffered saline (PBS) (pH 7.4) through the column at a flow rate of 1 ml/min, each eluted fraction (100 ug) was injected thereto so as to perform purity analysis. Purity refers to the purity (%) of the intact form and was calculated by the following equation.

Purity (%)=100−content of high molecular weight aggregates (%)

The results obtained by performing the purifications using a protein A affinity chromatography as described above are shown in Table 5 below.

TABLE 5

| | | | Purity | Yield |
|---|---|---|---|---|
| # | Elution buffer | Excipient | (%) | (%) |
| (1) | 100 mM sodium acetate buffer | — | 76.2 | 100 |
| | (pH 3.7) | | | |
| (2) | 100 mM sodium acetate buffer | CaCl₂ | 81.5 | 100 |
| | (pH 3.7) | (30 mM) | | |
| (3) | 100 mM sodium acetate buffer | CaCl₂ | 82.2 | 100 |
| | (pH 3.7) | (50 mM) | | |
| (4) | 100 mM sodium acetate buffer | CaCl₂ | 91.0 | 92.1 |
| | (pH 3.7) | (100 mM) | | |
| (5) | 100 mM sodium acetate buffer | CaCl₂ | 91.4 | 84.6 |
| | (pH 3.7) | (200 mM) | | |
| (6) | 100 mM sodium acetate buffer | CaCl₂ | 90.9 | 84.2 |
| | (pH 3.7) | (500 mM) | | |
| (7) | 20 mM sodium acetate buffer | CaCl₂ | 81.0 | 96.8 |
| | (pH 3.7) | (30 mM) | | |
| (8) | 20 mM sodium acetate buffer | CaCl₂ | 87.5 | 95.7 |
| | (pH 3.7) | (50 mM) | | |
| (9) | 20 mM sodium acetate buffer | CaCl₂ | 93.0 | 95.5 |
| | (pH 3.7) | (100 mM) | | |
| (10) | 20 mM sodium acetate buffer | CaCl₂ | 90.1 | 83.2 |
| | (pH 3.7) | (200 mM) | | |

As can be seen from the results in Table 5 above, when the sodium acetate buffers containing CaCl₂ were used as an excipient, the purity of anti-4-1BB/anti-HER2 bispecific antibody can be remarkably increased with a relatively high yield. And, from the results in Table 5, it can be seen that the concentration of CaCl₂ in the sodium acetate buffer is preferably in the range of 30 to 500 mM, more preferably in the range of 50 to 500 mM, still more preferably in the range of 75 to 125 mM, and particularly preferably about 100 mM. In addition, it can be seen that the concentration of sodium acetate in the sodium acetate buffer is preferably in the range of 20 to 100 mM.

### Comparative Example 1: Purification of Anti-4-1BB/Anti-HER2 Bispecific Antibody Using NaCl as an Excipient

Purification with protein A affinity chromatography was carried out in the same manner as in Example 1, using 100 mM sodium acetate buffer (pH3.7) containing NaCl at a concentration of 1000 mM as an excipient. For the eluted pool, the purity and yield were analyzed in the same manner as in Example 1. As the results thereof, the purity was relatively increased compared to the purification using 100 mM sodium acetate (pH 3.7) having no excipient, but the yield was significantly reduced to 29.3%.

From the above results, the purity and yield were evaluated by lowering the concentrations of sodium acetate and the excipient (NaCl). That is, purification with protein A affinity chromatography was carried out in the same manner as in Example 1, using 20 mM sodium acetate buffer (pH3.7) containing NaCl at a concentration of 100 mM as an excipient. For the eluted pool, the purity and yield were analyzed in the same manner as in Example 1. As the results thereof, the purity was significantly reduced to 74.2%.

Therefore, the use of a sodium acetate buffer containing NaCl did not show an improved purification effect, in terms of purity and yield.

### Comparative Example 2: Purification of Anti-4-1BB/Anti-HER2 Bispecific Antibody Using MgCl₂ as an Excipient

Purification with protein A affinity chromatography was carried out in the same manner as in Example 1, using 100 mM sodium acetate buffer (pH3.7) containing MgCl₂ at a concentration of 1000 mM as an excipient. For the eluted pool, the purity and yield were analyzed in the same manner as in Example 1. As the results thereof, the purity was relatively increased compared to the purification using 100 mM sodium acetate (pH 3.7) having no excipient, but the yield was significantly reduced to 43.9%.

From the above results, the purity and yield were evaluated by lowering the concentrations of sodium acetate and the excipient (MgCl₂). That is, purification with protein A affinity chromatography was carried out in the same manner as in Example 1, using 20 mM sodium acetate buffer (pH3.7) containing MgCl₂ at a concentration of 100 mM as an excipient. For the eluted pool, the purity and yield were analyzed in the same manner as in Example 1. As the results thereof, the purity was relatively increased compared to the purification as in the purification using a high concentration of sodium acetate/excipient, but the yield was only 66.7%.

Therefore, the use of a sodium acetate buffer containing MgCl₂ did not show an improved purification effect, in terms of yield.

1. A method for purifying an anti-4-1BB/anti-HER2 bispecific antibody, the method of which comprises (a) filtering a culture supernatant of an anti-4-1BB/anti-HER2 bispecific antibody-producing cell line to obtain a crude antibody-containing filtrate; (b) loading the filtrate onto a protein A affinity chromatography column; and (c) eluting the antibody from the column of step (b) with a sodium acetate buffer containing CaCl₂),
wherein
the heavy chain of the anti-4-1BB/anti-HER2 bispecific antibody comprises (i) a heavy chain of anti-HER2 antibody consisting of the amino acids of SEQ ID NO: 9 and (ii) a scFv of anti-4-1BB antibody comprising a light chain variable region of anti-4-1BB antibody comprising the amino acids of SEQ ID NOs: 1, 2, and 3; and a heavy chain variable region of anti-4-1BB antibody comprising the amino acids of SEQ ID NOs: 5, 6, and 7, and
the light chain of the anti-4-1BB/anti-HER2 bispecific antibody comprises a light chain of anti-HER2 antibody consisting of the amino acids of SEQ ID NO: 11.

2. The method according to claim 1, wherein the light chain variable region of anti-4-1BB antibody consists of the amino acids of SEQ ID NO: 4.

3. The method according to claim 1, wherein the heavy chain variable region of anti-4-1BB antibody consists of the amino acids of SEQ ID NO: 8.

4. The method according to claim 1, wherein the scFv of anti-4-1BB antibody further comprises a linker consisting of the amino acids of SEQ ID NO: 13.

5. The method according to claim 1, wherein the heavy chain of the anti-4-1BB/anti-HER2 bispecific antibody further comprises (iii) a linker consisting of the amino acids of SEQ ID NO: 12.

6. The method according to claim 1, wherein the heavy chain of the anti-4-1BB/anti-HER2 bispecific antibody consists of the amino acids of SEQ ID NO: 10.

7. The method according to claim 1, wherein CaCl₂) is present in a concentration ranging from 30 to 500 mM in the sodium acetate buffer.

8. The method according to claim 7, wherein CaCl₂) is present in a concentration ranging from 75 to 125 mM in the sodium acetate buffer.

9. The method according to claim 1, wherein sodium acetate is present in a concentration ranging from 20 to 100 mM in the sodium acetate buffer.

10. The method according to claim 1, wherein the sodium acetate buffer has a pH ranging from pH 3.5 to pH 4.0.

11. The method according to claim 1, wherein CaCl₂) is present in a concentration ranging from 75 to 125 mM in the sodium acetate buffer and sodium acetate is present in a concentration ranging from 20 to 100 mM in the sodium acetate buffer.

12. The method according to claim 11, wherein the sodium acetate buffer has a pH ranging from pH 3.5 to pH 4.0.
